(19) United States
(12) Patent Application Publication
(10) Pub. No.: US 2009/0036546 A1
(43) Pub. Date: Feb. 5, 2009

(54) Medicinal Oil Compositions, Preparations, and Applications Thereof

(75) Inventors: Ravindra Shah, Concord, CA (US); John M. Rosebaum, Richmond, CA (US); David C. Kramer, San Anselmo, CA (US); Alexander Munoz, Hercules, CA (US); Jan L. Arickx, Deinze (BE)

Correspondence Address: CHEVRON CORPORATION, P.O. BOX 6006, SAN RAMON, CA, 94583-0806, US

(73) Assignee: Chevron U.S.A. Inc., San Ramon, CA (US)

(21) Appl. No.: 11/831,923

(22) Filed: Jul. 31, 2007

(51) Int. Cl. A61K 31/01; A61P 43/00; C10M 169/04

(52) U.S. Cl. 514/762; 508/110

(57) ABSTRACT

A medicinal white oil composition is prepared from an isomerized base oil. The isomerized base oil is filtered through a filter bed containing an acid activated clay having a surface area of at least 100 m²/g, for the base oil to be in compliant with at least one of European Pharmacopeia 3^{rd} Edition, US Pharmacopeia 23^{rd} edition (USP), FDA 21 CFR 172.878 and FDA 21 CFR 178.3620(a) for direct food contact, and FDA 21 CFR 178.3570 (USDA H-1). The medicinal white oil in one embodiment has a UV absorbance at 260 to 350 nm of less than 0.1.

### TECHNICAL FIELD

The invention relates generally to medicinal white oil compositions, and more specifically to high performance medicinal white oil compositions comprising an isomerized base oil and applications/uses thereof.

### BACKGROUND

Personal/pharmaceutical products such as baby oils, shampoos, skin care products, etc., typically incorporate a certain amount of a lubricating oil additive that meets US Pharmacopoeia (USP) or European Pharmacopoeia (EP) specifications. The equipment used in the personal care products/pharmaceutical products/food processing industry may vary by segment/type of products being processed; however, the moving parts such as bearings, gears, and slide mechanisms are similar and often require lubrication. The most often used lubricants (“lubricating oils” or “oils”) include hydraulic, refrigeration, and gear oils as well as all-purpose greases. These oils must meet more stringent standards than other industry lubricants. They are classified as either H-1, H-2 or 3H by the USDA and NSF International. H-1 is for lubricants approved for incidental food contact. H-2 classification is for uses where there is no possibility of food contact and with no known poisons or carcinogens in the lubricant. 3H classification is for a release agent for food.

“Medicinal oil” or “medicinal white oil” herein refers to oil products meeting USP and/or EP specifications, and inherently, H-1 or 3H standards. Some medicinal white oil compositions in the prior art employ a white mineral oil in the base matrix. White mineral oils are prepared from a distillate of petroleum crude oil. The petroleum-based oils function satisfactorily, but they are not readily biodegradable. In other prior art embodiments, vegetable oils are used. However, many vegetable oils do not possess the desired pour point, oxidative stability, etc., among others properties of petroleum products.

Recent reforming processes have formed a new class of oil, e.g., Fischer Tropsch base oil (FTBO), wherein the oil, fraction, or feed originates from or is produced at some stage by a Fischer-Tropsch process. The feedstock for a Fischer-Tropsch process may come from a wide variety of hydrocarbonaceous resources, including biomass, natural gas, coal, shale oil, petroleum, municipal waste, derivatives of these, and combinations thereof. Crude product prepared from the Fischer-Tropsch process comprises a mixture of various solid, liquid, and gaseous hydrocarbons, which can be refined into products such as diesel oil, naphtha, wax, and other liquid petroleum or specialty products.

In a number of patent publications and applications, i.e., US2006/0289337, US2006/0201851, US2006/0016721, US2006/0016724, US2006/0076267, US2006/020185, US2006/013210, US2005/0241990, US2005/0077208, US2005/0139513, US2005/0139514, US2005/0133409, US2005/0133407, US2005/0261147, US2005/0261146, US2005/0261145, US2004/0159582, U.S. Pat. No. 7,018,525, U.S. Pat. No. 7,083,713, U.S. application Ser. Nos. 11/400570, 11/535165 and 11/613936, which are incorporated herein by reference, a Fischer Tropsch base oil is produced from a process in which the feed is a waxy feed recovered from a Fischer-Tropsch synthesis. The process comprises a complete or partial hydroisomerization dewaxing step, using a dual-functional catalyst or a catalyst that can isomerize paraffins selectively. Hydroisomerization dewaxing is achieved by contacting the waxy feed with a hydroisomerization catalyst in an isomerization zone under hydroisomerizing conditions.

U.S. Patent Publication No. 2006/0016721 discloses a white oil made from a Fischer Tropsch base oil, from a process wherein the yield of white oil boiling from 343° C. and above is greater than 25 wt % of the waxy feed and a viscosity at 100° C. of 1.5-36 mm2/s. WO2006/122979 discloses the use of a Fischer-Tropsch derived white oil in food contact applications, wherein the Fischer-Tropsch derived white oil has a kinematic viscosity at 100° C. of in between more than 2 mm²/s and less than 7 mm²/s according to ISO 3014, a content of mineral hydrocarbons with carbon numbers less than 25 of not more than 5% (w/w); and UV absorption spectra of <0.70 for 280-289 nm; <0.60 for 290-299 nm; <0.40 for 300-329 nm; and <0.09 for 300-380 nm.

There is still a need for a medicinal white oil having improved properties in compliance with FDA and USP requirements, e.g., exceptional UV absorbance properties as compared to the white oil made from Fischer Tropsch base oil of the prior art, Saybolt color, and additionally, having a combination of desired characteristics inherent to petroleum-based oils such as pour point and oxidative stability, and the biodegradability of vegetable-based base oils.

### SUMMARY OF THE INVENTION

In one aspect, there is provided a medicinal white oil composition in compliance with at least one of European Pharmacopeia 3^{rd} Edition, US Pharmacopeia 23^{rd} edition (USP), FDA 21 CFR 172.878 and FDA 21 CFR 178.3620(a) for direct food contact, FDA 21 CFR 178.3620(b) for indirect food contact, and FDA 21 CFR 178.3570 (USDA H-1) specifications, the composition is made by filtering an isomerized base oil having consecutive numbers of carbon atoms and less than 10 wt % naphthenic carbon by n-d-M through a filter bed containing an acid activated clay having a surface area of at least 100 m²/g. In one embodiment, the medicinal white oil has a UV absorbance at 260 to 50 nm of less than 0.1.

In another aspect, the invention relates to a method of preparing a medicinal white oil composition having a UV absorbance at 260 to 50 nm of less than 0.1, the method comprising filtering an isomerized base oil that through a filter bed containing an acid activated clay having a surface area of at least 100 m²/g, wherein the isomerized base oil has consecutive numbers of carbon atoms and less than 10 wt % naphthenic carbon by n-d-M.

### DETAILED DESCRIPTION

The following terms will be used throughout the specification and will have the following meanings unless otherwise indicated.

“Medicinal white oil” may be used interchangeably with medicinal oil, medicinal grade oil, or food grade oil, referring to an oil meeting at least one of the following standards: European Pharmacopeia 3^{rd} Edition; US Pharmacopeia 23^{rd} edition; FDA 21 CFR 172.878 and FDA 21 CFR 178.3620(a) for direct food contact; FDA 21 CFR 178.3620(b) for indirect food contact; and the lesser stringent FDA 21 CFR 178.3570 (USDA H-1) regulations for indirect food contact. In one embodiment, the medicinal white oil meets the test requirements of the United States Pharmacopeia (U.S.P.) XX (1980), at page 532, for readily carbonizable substances, and U.S.P. XVII for sulfur compounds at page 400. When used in a lubricating oil application, i.e., for lubricating food processing equipment that may come in contact with food, the medicinal oil is in compliance with USDA H-1 specification.

“Biodegradability” refers to the decrease in the amount of a substrate due to microbial action, conducted in accordance with CEC-L-33-T-82, a test method developed by the Coordinating European Council (CEC) and reported in “Biodegradability Of Two-Stroke Cycle Outboard Engine Oils In Water: Tentative Test Method” pp 1-8. Biodegradability can also be measured under OECD 301B, modified Sturm CO₂, which is a test method developed by the Organization for Economic Cooperation and Development and reported in “OECD Guidelines for the Testing of Chemicals,” Vol. 2, Section 3, pp. 18 24 (Adopted Jul. 17, 1992), measuring the aerobic microbial biodegradation of a test material by its complete breakdown to carbon dioxide. An oil is defined to be inherently biodegradable if the OECD 301B value is >=20%. The oil is readily biodegradable if its OECD 301B value is >=20%.

“RCS” refers to “readily carbonizable substances” which are impurities which cause an oil to change color when treated with strong acid. The US Food and Drug Administration (FDA) has stringent standards with respect to RCS. RCS is measured according to ASTM 565-99. RCS can also be measured according to the method specified in the USP Standards.

“Fischer-Tropsch derived” means that the product, fraction, or feed originates from or is produced at some stage by a Fischer-Tropsch process. As used herein, “Fischer-Tropsch base oil” may be used interchangeably with “FT base oil,” “FTBO,” “GTL base oil” (GTL: gas-to-liquid), or “Fischer-Tropsch derived base oil.”

As used herein, “isomerized base oil” refers to a base oil made by isomerization of a waxy feed.

As used herein, a “waxy feed” comprises at least 40 wt % n-paraffins. In one embodiment, the waxy feed comprises greater than 50 wt % n-paraffins. In another embodiment, greater than 75 wt % n-paraffins. In one embodiment, the waxy feed also has very low levels of nitrogen and sulphur, e.g., less than 25 ppm total combined nitrogen and sulfur, or in other embodiments less than 20 ppm. Examples of waxy feeds include slack waxes, deoiled slack waxes, refined foots oils, waxy lubricant raffinates, n-paraffin waxes, NAO waxes, waxes produced in chemical plant processes, deoiled petroleum derived waxes, microcrystalline waxes, Fischer-Tropsch waxes, and mixtures thereof In one embodiment, the waxy feeds have a pour point of greater than 50° C. In another embodiment, greater than 60° C.

“Kinematic viscosity” is a measurement in mm²/s of the resistance to flow of a fluid under gravity, determined by ASTM D445-06.

“Viscosity index” (VI) is an empirical, unit-less number indicating the effect of temperature change on the kinematic viscosity of the oil. The higher the VI of an oil, the lower its tendency to change viscosity with temperature. Viscosity index is measured according to ASTM D 2270-04.

Cold-cranking simulator apparent viscosity (CCS VIS) is a measurement in millipascal seconds, mPa.s to measure the viscometric properties of lubricating base oils under low temperature and high shear. CCS VIS is determined by ASTM D 5293-04.

The boiling range distribution of base oil, by wt%, is determined by simulated distillation (SIMDIS) according to ASTM D 6352-04, “Boiling Range Distribution of Petroleum Distillates in Boiling Range from 174 to 700° C. by Gas Chromatography.”

“Noack volatility” is defined as the mass of oil, expressed in weight %, which is lost when the oil is heated at 250° C. with a constant flow of air drawn through it for 60 min., measured according to ASTM D5800-05, Procedure B.

Brookfield viscosity is used to determine the internal fluid-friction of a lubricant during cold temperature operation, which can be measured by ASTM D 2983-04.

“Pour point” is a measurement of the temperature at which a sample of base oil will begin to flow under certain carefully controlled conditions, which can be determined as described in ASTM D 5950-02.

“Auto ignition temperature” is the temperature at which a fluid will ignite spontaneously in contact with air, which can be determined according to ASTM 659-78.

“Ln” refers to natural logarithm with base “e.”

“Traction coefficient” is an indicator of intrinsic lubricant properties, expressed as the dimensionless ratio of the friction force F and the normal force N, where friction is the mechanical force which resists movement or hinders movement between sliding or rolling surfaces. Traction coefficient can be measured with an MTM Traction Measurement System from PCS Instruments, Ltd., configured with a polished 19 mm diameter ball (SAE AISI 52100 steel) angled at 220 to a flat 46 mm diameter polished disk (SAE AISI 52100 steel). The steel ball and disk are independently measured at an average rolling speed of 3 meters per second, a slide to roll ratio of 40 percent, and a load of 20 Newtons. The roll ratio is defined as the difference in sliding speed between the ball and disk divided by the mean speed of the ball and disk, i.e. roll ratio=(Speed1−Speed2)/((Speed1+Speed2)−/2).

As used herein, “consecutive numbers of carbon atoms” means that the base oil has a distribution of hydrocarbon molecules over a range of carbon numbers, with every number of carbon numbers in-between. For example, the base oil may have hydrocarbon molecules ranging from C22 to C36 or from C30 to C60 with every carbon number in-between. The hydrocarbon molecules of the base oil differ from each other by consecutive numbers of carbon atoms, as a consequence of the waxy feed also having consecutive numbers of carbon atoms. For example, in the Fischer-Tropsch hydrocarbon synthesis reaction, the source of carbon atoms is CO and the hydrocarbon molecules are built up one carbon atom at a time. Petroleum-derived waxy feeds have consecutive numbers of carbon atoms. In contrast to an oil based on poly-alpha-olefin (“PAO”), the molecules of an isomerized base oil have a more linear structure, comprising a relatively long backbone with short branches. The classic textbook description of a PAO is a star-shaped molecule, and in particular tridecane, which is illustrated as three decane molecules attached at a central point. While a star-shaped molecules is theoretical, nevertheless PAO molecules have fewer and longer branches that the hydrocarbon molecules that make up the isomerized base oil disclosed herein.

“Molecules with cycloparaffinic functionality” mean any molecule that is, or contains as one or more substituents, a monocyclic or a fused multicyclic saturated hydrocarbon group.

“Molecules with monocycloparaffinic functionality” mean any molecule that is a monocyclic saturated hydrocarbon group of three to seven ring carbons or any molecule that is substituted with a single monocyclic saturated hydrocarbon group of three to seven ring carbons.

“Molecules with multicycloparaffinic functionality” mean any molecule that is a fused multicyclic saturated hydrocarbon ring group of two or more fused rings, any molecule that is substituted with one or more fused multicyclic saturated hydrocarbon ring groups of two or more fused rings, or any molecule that is substituted with more than one monocyclic saturated hydrocarbon group of three to seven ring carbons.

Molecules with cycloparaffinic functionality, molecules with monocycloparaffinic functionality, and molecules with multicycloparaffinic functionality are reported as weight percent and are determined by a combination of Field Ionization Mass Spectroscopy (FIMS), HPLC-UV for aromatics, and Proton NMR for olefins, further fully described herein.

Oxidator BN measures the response of a lubricating oil in a simulated application. High values, or long times to adsorb one liter of oxygen, indicate good stability. Oxidator BN can be measured via a Dornte-type oxygen absorption apparatus (R. W. Dornte “Oxidation of White Oils,” Industrial and Engineering Chemistry, Vol. 28, page 26, 1936), under 1 atmosphere of pure oxygen at 340° F., time to absorb 1000 ml of O₂ by 100 g. of oil is reported. In the Oxidator BN test, 0.8 ml of catalyst is used per 100 grams of oil. The catalyst is a mixture of soluble metal-naphthenates simulating the average metal analysis of used crankcase oil. The additive package is 80 millimoles of zinc bispolypropylenephenyldithiophosphate per 100 grams of oil.

Molecular characterizations can be performed by methods known in the art, including Field Ionization Mass Spectroscopy (FIMS) and n-d-M analysis (ASTM D 3238-95 (Re-approved 2005)). In FIMS, the base oil is characterized as alkanes and molecules with different numbers of unsaturations. The molecules with different numbers of unsaturations may be comprised of cycloparaffins, olefins, and aromatics. If aromatics are present in significant amount, they would be identified as 4-unsaturations. When olefins are present in significant amounts, they would be identified as 1-unsaturations. The total of the 1-unsaturations, 2-unsaturations, 3-unsaturations, 4-unsaturations, 5-unsaturations, and 6-unsaturations from the FIMS analysis, minus the wt % olefins by proton NMR, and minus the wt % aromatics by HPLC-UV is the total weight percent of molecules with cycloparaffinic functionality. If the aromatics content was not measured, it was assumed to be less than 0.1 wt % and not included in the calculation for total weight percent of molecules with cycloparaffinic functionality. The total weight percent of molecules with cycloparaffinic functionality is the sum of the weight percent of molecules with monocyclopraffinic functionality and the weight percent of molecules with multicycloparaffinic functionality.

Molecular weights are determined by ASTM D2503-92(Reapproved 2002). The method uses thermoelectric measurement of vapour pressure (VPO). In circumstances where there is insufficient sample volume, an alternative method of ASTM D2502-94 may be used; and where this has been used it is indicated.

Density is determined by ASTM D4052-96 (Reapproved 2002). The sample is introduced into an oscillating sample tube and the change in oscillating frequency caused by the change in the mass of the tube is used in conjunction with calibration data to determine the density of the sample.

Weight percent olefins can be determined by proton-NMR according to the steps specified herein. In most tests, the olefins are conventional olefins, i.e. a distributed mixture of those olefin types having hydrogens attached to the double bond carbons such as: alpha, vinylidene, cis, trans, and trisubstituted, with a detectable allylic to olefin integral ratio between 1 and 2.5. When this ratio exceeds 3, it indicates a higher percentage of tri or tetra substituted olefins being present, thus other assumptions known in the analytical art can be made to calculate the number of double bonds in the sample. The steps are as follows: A) Prepare a solution of 5-10% of the test hydrocarbon in deuterochloroform. B) Acquire a normal proton spectrum of at least 12 ppm spectral width and accurately reference the chemical shift (ppm) axis, with the instrument having sufficient gain range to acquire a signal without overloading the receiver/ADC, e.g., when a 30 degree pulse is applied, the instrument having a minimum signal digitization dynamic range of 65,000. In one embodiment, the instrument has a dynamic range of at least 260,000. C) Measure the integral intensities between: 6.0-4.5 ppm (olefin); 2.2-1.9 ppm (allylic); and 1.9-0.5 ppm (saturate). D) Using the molecular weight of the test substance determined by ASTM D 2503-92 (Reapproved 2002), calculate: 1. The average molecular formula of the saturated hydrocarbons; 2. The average molecular formula of the olefins; 3. The total integral intensity (=sum of all integral intensities); 4. The integral intensity per sample hydrogen (=total integral/number of hydrogens in formula); 5. The number of olefin hydrogens (=olefin integral/integral per hydrogen); 6. The number of double bonds (=olefin hydrogen times hydrogens in olefin formula/2); and 7. The wt % olefins by proton NMR=100 times the number of double bonds times the number of hydrogens in a typical olefin molecule divided by the number of hydrogens in a typical test substance molecule. In this test, the wt % olefins by proton NMR calculation procedure, D, works particularly well when the percent olefins result is low, less than 15 wt %.

Weight percent aromatics in one embodiment can be measured by HPLC-UV. In one embodiment, the test is conducted using a Hewlett Packard 1050 Series Quaternary Gradient High Performance Liquid Chromatography (HPLC) system, coupled with a HP 1050 Diode-Array UV-Vis detector interfaced to an HP Chem-station. Identification of the individual aromatic classes in the highly saturated base oil can be made on the basis of the UV spectral pattern and the elution time. The amino column used for this analysis differentiates aromatic molecules largely on the basis of their ring-number (or double-bond number). Thus, the single ring aromatic containing molecules elute first, followed by the polycyclic aromatics in order of increasing double bond number per molecule. For aromatics with similar double bond character, those with only alkyl substitution on the ring elute sooner than those with naphthenic substitution. Unequivocal identification of the various base oil aromatic hydrocarbons from their UV absorbance spectra can be accomplished recognizing that their peak electronic transitions are all red-shifted relative to the pure model compound analogs to a degree dependent on the amount of alkyl and naphthenic substitution on the ring system. Quantification of the eluting aromatic compounds can be made by integrating chromatograms made from wavelengths optimized for each general class of compounds over the appropriate retention time window for that aromatic. Retention time window limits for each aromatic class can be determined by manually evaluating the individual absorbance spectra of eluting compounds at different times and assigning them to the appropriate aromatic class based on their qualitative similarity to model compound absorption spectra.

HPLC-UV Calibration. In one embodiment, HPLC-UV can be used for identifying classes of aromatic compounds even at very low levels, e.g., multi-ring aromatics typically absorb 10 to 200 times more strongly than single-ring aromatics. Alkyl-substitution affects absorption by 20%. Integration limits for the co-eluting 1-ring and 2-ring aromatics at 272 nm can be made by the perpendicular drop method. Wavelength dependent response factors for each general aromatic class can be first determined by constructing Beer's Law plots from pure model compound mixtures based on the nearest spectral peak absorbances to the substituted aromatic analogs. Weight percent concentrations of aromatics can be calculated by assuming that the average molecular weight for each aromatic class was approximately equal to the average molecular weight for the whole base oil sample.

NMR analysis. In one embodiment, the weight percent of all molecules with at least one aromatic function in the purified mono-aromatic standard can be confirmed via long-duration carbon 13 NMR analysis. The NMR results can be translated from % aromatic carbon to % aromatic molecules (to be consistent with HPLC-UV and D 2007) knowing that 95-99% of the aromatics in highly saturated base oils are single-ring aromatics. In another test to accurately measure low levels of all molecules with at least one aromatic function by NMR, the standard D 5292-99 (Reapproved 2004) method can be modified to give a minimum carbon sensitivity of 500:1 (by ASTM standard practice E 386) with a 15-hour duration run on a 400-500 MHz NMR with a 10-12 mm Nalorac probe. Acorn PC integration software can be used to define the shape of the baseline and consistently integrate.

Extent of branching refers to the number of alkyl branches in hydrocarbons. Branching and branching position can be determined using carbon-13 (¹³C) NMR according to the following nine-step process: 1) Identify the CH branch centers and the CH₃ branch termination points using the DEPT Pulse sequence (Doddrell, D. T.; D. T. Pegg; M. R. Bendall, Journal of Magnetic Resonance 1982, 48, 323ff.). 2) Verify the absence of carbons initiating multiple branches (quaternary carbons) using the APT pulse sequence (Patt, S. L.; J. N. Shoolery, Journal of Magnetic Resonance 1982, 46, 535ff.). 3) Assign the various branch carbon resonances to specific branch positions and lengths using tabulated and calculated values known in the art (Lindeman, L. P., Journal of Qualitative Analytical Chemistry 43, 1971 1245ff; Netzel, D. A., et al., Fuel, 60, 1981, 307ff). 4) Estimate relative branching density at different carbon positions by comparing the integrated intensity of the specific carbon of the methyl/alkyl group to the intensity of a single carbon (which is equal to total integral/number of carbons per molecule in the mixture). For the 2-methyl branch, where both the terminal and the branch methyl occur at the same resonance position, the intensity is divided by two before estimating the branching density. If the 4-methyl branch fraction is calculated and tabulated, its contribution to the 4+methyls is subtracted to avoid double counting. 5) Calculate the average carbon number. The average carbon number is determined by dividing the molecular weight of the sample by 14 (the formula weight of CH₂). 6) The number of branches per molecule is the sum of the branches found in step 4. 7) The number of alkyl branches per 100 carbon atoms is calculated from the number of branches per molecule (step 6) times 100/average carbon number. 8) Estimate Branching Index (BI) by ¹H NMR Analysis, which is presented as percentage of methyl hydrogen (chemical shift range 0.6-1.05 ppm) among total hydrogen as estimated by NMR in the liquid hydrocarbon composition. 9) Estimate Branching proximity (BP) by ¹³C NMR, which is presented as percentage of recurring methylene carbons—which are four or more carbons away from the end group or a branch (represented by a NMR signal at 29.9 ppm) among total carbons as estimated by NMR in the liquid hydrocarbon composition. The measurements can be performed using any Fourier Transform NMR spectrometer, e.g., one having a magnet of 7.0 T or greater. After verification by Mass Spectrometry, UV or an NMR survey that aromatic carbons are absent, the spectral width for the ¹³C NMR studies can be limited to the saturated carbon region, 0-80 ppm vs. TMS (tetramethylsilane). Solutions of 25-50 wt. % in chloroform-d1 are excited by 30 degrees pulses followed by a 1.3 sec acquisition time. In order to minimize non-uniform intensity data, the broadband proton inverse-gated decoupling is used during a 6 sec delay prior to the excitation pulse and on during acquisition. Samples are doped with 0.03 to 0.05 M Cr (acac)₃ (tris (acetylacetonato)-chromium (III)) as a relaxation agent to ensure full intensities are observed. The DEPT and APT sequences can be carried out according to literature descriptions with minor deviations described in the Varian or Bruker operating manuals. DEPT is Distortionless Enhancement by Polarization Transfer. The DEPT 45 sequence gives a signal all carbons bonded to protons. DEPT 90 shows CH carbons only. DEPT 135 shows CH and CH₃ up and CH₂ 180 degrees out of phase (down). APT is attached proton test, known in the art. It allows all carbons to be seen, but if CH and CH₃ are up, then quaternaries and CH₂ are down. The branching properties of the sample can be determined by ¹³C NMR using the assumption in the calculations that the entire sample was iso-paraffinic. The unsaturates content may be measured using Field Ionization Mass Spectroscopy (FIMS).

In one embodiment, the medicinal white oil composition comprises an isomerized base oil that has been filtered through a clay absorbent.

Isomerized Base Oil Component: In one embodiment, the medicinal white oil composition consists essentially of an isomerized base oil as the base material. As used herein, the expression “consisting essentially of” permits the inclusion of components that do not materially affect the basic and novel characteristics of the composition under consideration.

In one embodiment, the base oil or blends thereof comprises at least an isomerized base oil which the product itself, its fraction, or feed originates from or is produced at some stage by isomerization of a waxy feed from a Fischer-Tropsch process (“Fischer-Tropsch derived base oils”). In another embodiment, the base oil comprises at least an isomerized base oil made from a substantially paraffinic wax feed (“waxy feed”).

Fischer-Tropsch derived base oils are disclosed in a number of patent publications, including for example U.S. Pat. Nos. 6,080,301, 6,090,989, and 6,165,949, and US Patent Publication No. US2004/0079678A1, US20050133409, US20060289337. Fischer-Tropsch process is a catalyzed chemical reaction in which carbon monoxide and hydrogen are converted into liquid hydrocarbons of various forms including a light reaction product and a waxy reaction product, with both being substantially paraffinic.

Fischer-Tropsch derived base oils are disclosed in a number of patent publications, including for example U.S. Pat. Nos. 6,080,301, 6,090,989, and 6,165,949, and US Patent Publication No. US2004/0079678A1, US20050133409, US20060289337. Fischer-Tropsch process is a catalyzed chemical reaction in which carbon monoxide and hydrogen are converted into liquid hydrocarbons of various forms including a light reaction product and a waxy reaction product, with both being substantially paraffinic.

In one embodiment the isomerized base oil has consecutive numbers of carbon atoms and has less than 10 wt % naphthenic carbon by n-d-M. In yet another embodiment the isomerized base oil made from a waxy feed has a kinematic viscosity at 100° C. between 1.5 and 3.5 mm²/s.

In one embodiment, the isomerized base oil is made by a process in which the hydroisomerization dewaxing is performed at conditions sufficient for the base oil to have: a) a weight percent of all molecules with at least one aromatic functionality less than 0.30; b) a weight percent of all molecules with at least one cycloparaffinic functionality greater than 10; c) a ratio of weight percent molecules with monocycloparaffinic functionality to weight percent molecules with multicycloparaffinic functionality greater than 20 and d) a viscosity index greater than 28×Ln (Kinematic viscosity at 100° C.)+80.

In another embodiment, the isomerized base oil is made from a process in which the highly paraffinic wax is hydroisomerized using a shape selective intermediate pore size molecular sieve comprising a noble metal hydrogenation component, and under conditions of 600-750° F. (315-399° C.) In the process, the conditions for hydroisomerization are controlled such that the conversion of the compounds boiling above 700° F. (371° C.) in the wax feed to compounds boiling below 700° F. (371° C.) is maintained between 10 wt % and 50 wt %. A resulting isomerized base oil has a kinematic viscosity of between 1.0 and 3.5 mm²/s at 100° C. and aNoack volatility of less than 50 weight %. The base oil comprises greater than 3 weight % molecules with cycloparaffinic functionality and less than 0.30 weight percent aromatics.

In one embodiment the isomerized base oil has a Noack volatility less than an amount calculated by the following equation: 1000×(Kinematic Viscosity at 100° C.)⁻²⁷. In another embodiment, the isomerized base oil has a Noack volatility less than an amount calculated by the following equation: 900×(Kinematic Vicosity at 100° C.)^{-2.1}. In a third embodiment, the isomerized base oil has a Kinematic Vicosity at 100° C. of >1.808 mm²/s and a Noack volatility less than an amount calculated by the following equation: 1.286+20 (kv100)^{-1.5}+551.8 e^{-kv100}, where kv100 is the kinematic viscosity at 100° C. In a fourth embodiment, the isomerized base oil has a kinematic viscosity at 100° C. of less than 4.0 mm²/s, and a wt % Noack volatility between 0 and 100. In a fifth embodiment, the isomerized base oil has a kinematic viscosity between 1.5 and 4.0 mm²/s and a Noack volatility less than the Noack volatility calculated by the following equation: 160-40 (Kinematic Viscosity at 100° C.).

In one embodiment, the isomerized base oil has a kinematic viscosity at 100° C. in the range of 2.4 and 3.8 mm²/s and a Noack volatility less than an amount defined by the equation: 900×(Kinematic Viscosity at 100° C.)^{-2.8}−15). For kinematic viscosities in the range of 2.4 and 3.8 mm²/s the equation: 900×(Kinematic Viscosity at 100° C.)^{-2.8}−15) provides a lower Noack volatility than the equation: 160-40 (Kinematic Viscosity at 100° C.)

In one embodiment, the isomerized base oil is made from a process in which the highly paraffinic wax is hydroisomerized under conditions for the base oil to have a kinematic viscosity at 100° C. of 3.6 to 4.2 mm²/s, a viscosity index of greater than 130, a wt % Noack volatility less than 12, a pour point of less than −9° C.

In one embodiment, the isomerized base oil has an auto-ignition temperature (AIT) greater than the AIT defined by the equation: AIT in ° C. =1.6×(Kinematic Viscosity at 40° C., in mm²/s)+300. In a second embodiment, the base oil as an AIT of greater than 329° C. and a viscosity index greater than 28×Ln (Kinematic Viscosity at 100° C., in mm²/s)+100.

In one embodiment, the isomerized base oil has a relatively low traction coefficient, specifically, its traction coefficient is less than an amount calculated by the equation: traction coefficient=0.009×Ln (kinematic viscosity in mm²/S)−0.001, wherein the kinematic viscosity in the equation is the kinematic viscosity during the traction coefficient measurement and is between 2 and 50 mm²/s. In one embodiment, the isomerized base oil has a traction coefficient of less than 0.023 (or less than 0.021) when measured at a kinematic viscosity of 15 mm²/s and at a slide to roll ratio of 40%. In another embodiment the isomerized base oil has a traction coefficient of less than 0.017 when measured at a kinematic viscosity of 15 mm²/s and at a slide to roll ratio of 40%. In another embodiment the isomerized base oil has a viscosity index greater than 150 and a traction coefficient less than 0.015 when measured at a kinematic viscosity of 15 mm²/s and at a slide to roll ratio of 40 percent.

In some embodiments, the isomerized base oil having low traction coefficients also displays a higher kinematic viscosity and higher boiling points. In one embodiment, the base oil has a traction coefficient less than 0.015, and a 50 wt % boiling point greater than 565° C. (1050° F.). In another embodiment, the base oil has a traction coefficient less than 0.011 and a 50 wt % boiling point by ASTM D 6352-04 greater than 582° C. (1080° F.).

In some embodiments, the isomerized base oil having low traction coefficients also displays unique branching properties by NMR, including a branching index less than or equal to 23.4, a branching proximity greater than or equal to 22.0, and a Free Carbon Index between 9 and 30. In one embodiment, the base oil has at least 4 wt % naphthenic carbon, in another embodiment, at least 5 wt % naphthenic carbon by n-d-M analysis by ASTM D 3238-95 (Reapproved 2005).

In one embodiment, the isomerized base oil is produced in a process wherein the intermediate oil isomerate comprises paraffinic hydrocarbon components, and in which the extent of branching is less than 7 alkyl branches per 100 carbons, and wherein the base oil comprises paraffinic hydrocarbon components in which the extent of branching is less than 8 alkyl branches per 100 carbons and less than 20 wt % of the alkyl branches are at the 2 position. In one embodiment, the FT base oil has a pour point of less than −8° C.; a kinematic viscosity at 100° C. of at least 3.2 mm²/s; and a Viscosity Index greater than a viscosity index calculated by the equation of =22×Ln (Kinematic Viscosity at 100° C.)+132.

In one embodiment, the base oil comprises greater than 10 wt. % and less than 70 wt. % total molecules with cycloparaffinic functionality, and a ratio of weight percent molecules with monocycloparaffinic functionality to weight percent molecules with multicycloparaffinic functionality greater than 15.

In one embodiment, the isomerized base oil has an average molecular weight between 600 and 1100, and an average degree of branching in the molecules between 6.5 and 10 alkyl branches per 100 carbon atoms. In another embodiment, the isomerized base oil has a kinematic viscosity between about 8 and about 25 mm²/s and an average degree of branching in the molecules between 6.5 and 10 alkyl branches per 100 carbon atoms.

In one embodiment, the isomerized base oil is obtained from a process in which the highly paraffinic wax is hydroisomerized at a hydrogen to feed ratio from 712.4 to 562 liter H₂/liter oil, for the base oil to have a total weight percent of molecules with cycloparaffinic functionality of greater than 10, and a ratio of weight percent molecules with monocycloparaffinic functionality to weight percent molecules with multicycloparaffinic functionality of greater than 15. In another embodiment, the base oil has a viscosity index greater than an amount defined by the equation: 28×Ln (Kinematic viscosity at 100° C.)+95. In a third embodiment, the base oil comprises a weight percent aromatics less than 0.30; a weight percent of molecules with cycloparaffinic functionality greater than 10; a ratio of weight percent of molecules with monocycloparaffinic functionality to weight percent of molecules with multicycloparaffinic functionality greater than 20; and a viscosity index greater than 28×Ln (Kinematic Viscosity at 100° C.)+110. In a fourth embodiment, the base oil further has a kinematic viscosity at 100° C. greater than 6 mm²/s. In a fifth embodiment, the base oil has a weight percent aromatics less than 0.05 and a viscosity index greater than 28×Ln (Kinematic Viscosity at 100° C.)+95. In a sixth embodiment, the base oil has a weight percent aromatics less than 0.30, a weight percent molecules with cycloparaffinic functionality greater than the kinematic viscosity at 100° C., in mm²/s, multiplied by three, and a ratio of molecules with monocycloparaffinic functionality to molecules with multicycloparaffinic functionality greater than 15.

In one embodiment, the isomerized base oil contains between 2 and 10% naphthenic carbon as measured by n-d-M. In one embodiment, the base oil has a kinematic viscosity of 1.5-3.0 mm²/s at 100° C. and 2-3% naphthenic carbon. In another embodiment, a kinematic viscosity of 1.8-3.5 mm²/s at 100° C. and 2.5-4% naphthenic carbon. In a third embodiment, a kinematic viscosity of 3-6 mm²/s at 100° C. and 2.7-5% naphthenic carbon. In a fourth embodiment, a kinematic viscosity of 10-30 mm²/s at 100° C. and greater than 5.2% naphthenic carbon.

In one embodiment, the isomerized base oil has an average molecular weight greater than 475; a viscosity index greater than 140, and a weight percent olefins less than 10. The base oil improves the air release and low foaming characteristics of the mixture when incorporated into the medicinal oil composition.

In one embodiment, the isomerized base oil is a white oil as disclosed in U.S. Pat. No. 7,214,307 and US Patent Publication US20060016724. In one embodiment, the isomerized white base oil has a kinematic viscosity at 100° C. between about 1.5-36 mm²/s; a viscosity index greater than an amount calculated by the equation: Viscosity Index=28×Ln(the Kinematic Viscosity at 100° C.)+105, less than 18 wt. % molecules with cycloparaffinic functionality, a pour point less than 0° C., and a Saybolt color of +20 or greater. In another embodiment, the isomerized base oil is a white oil having a kinematic viscosity at 100° C. between about 1.5 cSt and 36 mm²/s, a viscosity index greater than an amount calculated by the equation: Viscosity Index=28×Ln(the Kinematic Viscosity at 100° C.)+95, between 5 and less than 18 weight percent molecules with cycloparaffinic functionality, less than 1.2 weight percent molecules with multicycloparaffinic functionality, a pour point less than 0° C. and a Saybolt color of +20 or greater.

Depending on the application, e.g., whether the medicinal oil is used for a wide temperature range application, a heavy-duty application, applications requiring water resistant, etc., different types of oils can be used. In one embodiment, the base oil has a kinematic viscosity at 100° C. between 1.5-3.0 mm²/s, or between 1.8-2.3 mm²/s. In another embodiment, a kinematic viscosity at 100° C. between 1.8-3.5 mm²/s, or between 2.3-3.5 mm²/s. In a third embodiment, a kinematic viscosity at 100° C. between 3.0-7.0 mm²/s, or between 3.5-5.5 mm²/s. In a fourth embodiment, a kinematic viscosity at 100° C. between 5.0-15.0 mm²/s, or between 5.5-10.0 mm²/s. In a fifth embodiment, a kinematic viscosity at 100° C. above 10 mm²/s. In a sixth embodiment, the base oil has a kinematic viscosity at 100° C. between 10.0-30.0 mm²/s, or between 15.0-30.0 mm²/s. In a seventh embodiment, the base oil matrix has a kinematic viscosity in the range of 5-400 mm²/s at 40° C. In an eight embodiment, the base oil matrix has a kinematic viscosity ranging from 10-200 mm²/s at 40° C. In yet another embodiment, the Fischer-Tropsch derived base oil has a viscosity of between about 3-9 mm²/s at 100° C.; a TGA Noack volatility of less than 35 wt. %; an initial boiling point within the range of between about 550-625° F.; an end boiling point between about 1000-1400° F.; and wherein less than 20 wt. % of the blend boils within the region defined by the 50 wt. % plus or minus 25° F.

In one embodiment, the isomerized base oil has a kinematic viscosity at 100° C. between 2 mm²/s and 30 mm²/s; a kinematic viscosity at 40° C. between 6 and 120 mm²/s; a viscosity index between 120 and 170; cold cranking simulator viscosity in the range of 2,000-25,000 at −25° C., 1,000-10,000 at −20° C., 500 and 5,000 at −15° C., and 1,000 and 3,500 at −10° C.; pour point in the range of −50 and −1° C.; molecular weight by ASTM D6503 of 300-800; density in the range of 0.7970 to 0.8400; refractive index of 1.440 to 1.47; paraffinic carbon in the range of 90-98%; naphthenic carbon in the range of 2-10%; oxidator BN of 30 to 70 hours; bromine index of 12 to 60. In another embodiment, the isomerized base oil has a TGA Noack in wt. % of 0.70 to 67 as measured by ASTM D5800-05 Procedure B. In yet another embodiment, the isomerized base oil has a ratio of weight percent molecules with monocycloparaffinic functionality to weight percent molecules with multicycloparaffinic functionality in the range of 2 to 25.

In one embodiment, the isomerized base oil has a kinematic viscosity at 100° C. between 3 mm²/s and 10 mm²/s; a kinematic viscosity at 40° C. between 15 and 50 mm²/s; a viscosity index between 130 and 160; and pour point between −20 and −20° C.

Additional Components: The European Pharmacopoeia (EP) does not allow the addition of additives to medicinal oils. The US Pharmanacopoeia (USP), however, allows for the addition of antioxidants such as Vitamin E, recommended for countries with hot climates wherein the white oils are at risk of oxidation. Depending on the final application, e.g., whether for use as a USP/EP grade medicinal oil in a pharmaceutical/personal care product formulation in Europe, or as a lubricating product meeting H-1 specifications to protect and lubricate chains, parts, etc. in the food industry in the US, additional components may or may not be added to the medicinal oil composition in an amount not greater than that required to produce its intended effect.

In one embodiment, up to 20 wt. % of a base oil thickener component such as food grade polybutene and/or food grade hydrotreated polybutene can be added to the base oil in a sufficient amount to adjust the product viscosity while maintaining a high quality viscosity index (VI). Thickeners such as food grade polybutene can be injected into the base oil as a smaller dose of a high viscosity material such as Indopol H1500 Polybutene (121,000 cSt at 40° C. and 3000 cSt at 100° C.) or as a larger dose of a lower viscosity material such as Indopol L-14 (27 CS at 40° C. and 4.7 cSt at 100° C.), both from Amoco Chemical Company. Smaller doses of higher viscosity polybutene such as H1500 provide better antioxidation results and are preferred over larger doses of lower viscosity polybutene (L-14).

In one embodiment, an amount of at least an extreme pressure (EP) additive is added to the medicinal oil. Examples include, but are not limited to, a phosphate ester oil additive, in amounts ranging from 0.01% and 25.00% by volume.

In another embodiment, a suitable non-toxic antioxidant in an amount of 0.05 to 2.0 wt. % is added to de-toxify the EP additive. An example is a biological antioxidant such as DL-alpha-Tocopherol, U.S.P./N.F. (CAS#59-02-9), of the vitamin E group, in an amount of 26.0 grams per 54.0 gallons of the base oil fluid.

In yet another embodiment, other antioxidants can be used. Examples include food grade, oil-soluble, sterically hindered phenols and thiophenols, e.g., sterically hindered phenolics such as hindered phenols and bis-phenols, hindered 4,4′-thiobisphenols, hindered 4-hydroxy-and 4-thiolbenzoic acid esters and dithio esters, and hindered bis(4-hydroxy-and 4-thiolbenzoic acid and dithio acid)alkylene esters. In one embodiment, the antioxidant is selected from the group of food grade, oil-soluble aromatic amine antioxidants are naphthyl phenyl amines, alkylated phenyl naphthyl amines, and alkylated diphenyl amines. In one embodiment, the composition comprises phenolic and aromatic amine antioxidants in a ratio by weight ranging from 20:1 to 1:20.

Depending on the application, in one embodiment, the medicinal oil composition comprises at least an anti-rust additive package having a combination of food grade ionic and non-ionic surface active anti-rust ingredients in an amount of 0.05 to 2.0 wt. %. Examples of ionic anti-rust lubricating additives include food grade phosphoric acid, mono and dihexyl ester compounds with tetramethyl nonyl amines, and mixtures thereof. Examples of non-ionic anti-rust lubricating additives include food grade fatty acids and their esters formed from the addition of sorbitan, glycerol, or other polyhydric alcohols, or polyalkylene glycols. Other non-ionic anti-rust lubricating additives can include food grade ethers from fatty alcohols alkoxylated with alkylene oxides, or sorbitan alkoxylated with alkylene oxides, or sorbitan esters alkoxylated with alkylene oxides.

Depending on the application, in one embodiment, the composition comprises at least an anti-wear additive. Examples include but are not limited to food grade oil-soluble sulfur and/or phosphorus containing compounds such as a triphenyl phosphorothioate. Other sulfur and/or phosphorus containing materials which are not currently approved for food grade use include: zinc dialkyl dithiophosphate, zinc dithiocarbamate, amine dithiocarbamate, and methylene bis dithiocarbamate. Any of the above compounds, with H-1 approval, would be a suitable anti-wear additive.

In one embodiment, the composition further comprises a suitable non-toxic emulsifier in an amount sufficient to completely emulsify the mixture. Examples include polyoxypropylene 15 stearyl ether (CFTA name: PPG-15 Stearyl Ether); ARLAMOL E Emollient-Solvent, available from ICI Surfactants; U.S.P./N.F. Grade emulsifying agents such as Acacia (CAS#9000-01-5); 2-Aminoethanol (CAS#141-43-5); Cholesterol (CAS#57-88-5); Octadecanoic Acid (CAS#57-11-4); lecithin; 9-Octadecanoic Acid (CAS#112-80-1); Polyethylene-Polypropylene Glycol (CAS#9003-11-6); Polyoxyl 20Cetostearyl Ester (CAS#9005-00-9); Polyoxyl 40 Stearyl (CAS#9004-99-3); Polysorbate 20(CAS#9005-64-5); Polysorbate 40(CAS#9005-66-7); Polysorbate 60 (CAS#9005-67-8); Polysorbate 80(CAS#9005-65-8); Sodium Lauryl Sulfate (CAS#151-21-3); Sodium Stearate (CAS#882-162); Sorbitan Monooleate (CAS#1338-43-8); Sorbitan Monopalmitate (CAS#26266-57-9); Sorbitan Monostearate (CAS#1338-41-6); Triethanolamine (CAS#102-71-6).

In one embodiment, following the blending of the antioxidant and emulsifier substituents into the base fluid of the medicinal oil composition, the mixture is buffered so as to be physiologically neutral, pH 7.3-7.48. A suitable buffering agent is acetic acid, 36% (w/w), U.S.P./N.F. (CAS#64-19-7). In one embodiment, an appropriate non-toxic antimicrobial compound is added in then an appropriate efficacious amount to produce the final mixture, between about 0.01% and 25.00% by volume of the final mixture. A suitable antimicrobial compound could be selected from the following group: Chlorhexidine gluconate (CAS#18472-51-0); Cetylpyridinium chloride (CAS#123-03-5); Sanguinarine (CAS#244754-3); Sodium fluoride (CAS#7681-49-4); Thymol (CAS#89-83-8); and equal parts of: (a) Alkyl dimethyl betaine (CAS#693-33-4) and (b) N,N-dimethyl alkylamine-N-oxide (CAS#3332-27-2). The type and amount of the non-toxic antimicrobial compound to be added would depend on the variety of microorganisms to be controlled, such as fungus, bacteria, algae, viruses and yeast, but not necessarily limited to these varieties. The relative amounts of antimicrobial compounds to be added to the final mixture will depend on the application and the useful antimicrobial dosage range for a particular application. Typical such applications would include equipment used in the processing and/or manufacturing of health care products, dental instruments and/or the processing and/or manufacturing of dental care products, equipment used in and/or manufacturing of food processing systems, equipment used in the processing and/or manufacturing of cosmetic and/or pharmaceutical products and any other of the like.

Method for Making: Prior to the addition of additives, if any, the isomerized base oil is first treated to meet the requirements for a medicinal white oil. In one embodiment, the isomerized base oil is filtered through a filter bed with absorbent containing clay or clay-like for removing precursors that can cause the base oil to fail the UV absorbent test, including but not limited to RCS precursors such as single and double ring aromatics and olefins.

In one embodiment, the clay is non-regenerable, i.e., the material is not easily, at least to an economically attractive extent, regenerated by solvent washing, by heating and/or by other methods known in the art for removing the contaminant load from the sorbent and returning the sorbent to its desired activity and capacity for preparing the white oil product. In another embodiment, a clay containing a zeolite is used. In another embodiment, the sorbent is an acid-activated clay. In yet another embodiment, the adsorbent is an acid-activated clay having a surface area of at least 100 m²/g. In a fourth embodiment, the adsorbent has a surface area of at least 150 m²/g. In a fourth embodiment, the adsorbent has a surface area of at least 200 m²/g.

Examples include acid-activated clays described in D. R. Taylor and D. B. Jenkins, Acid-activated Clays, Society of Mining Engineers of AIME (Transactions), vol 282, p. 1901-1910. An acid-activated clay is defined as a nonswelling bentonite that has been treated with mineral acid to enhance its capacity for adsorbing pigments from oils. A bentonite is a clay ore whose principal mineral in montmorillonite, an end-member of the smectite clay mineral group characterized by a three-layered structure composed of two silica sheets sandwiches about a central alumina sheet. In yet another embodiment, the clay is an intercalating clay mineral acid activated in the presence of a polar organic liquid such as an aliphatic C₁ to C₆ monohydric alcohol or an aliphatic C₂ to C₆ aliphatic ether, as disclosed in U.S. Pat. No. 5,908,500.

In one embodiment, the isomerized base oil is filtered through a filter bed with an acid-activated calcium bentonite clay as the sorbent. Examples of acid activated clays commercially available include TONSIL CO 630G, Tonsil Optimum 320 FF, Tonsil L-80, activated clay from Sud-Chemie Indonesia, and Activated Bleaching Clay from HRP Industries of India, etc.

In one embodiment, the sorbent material for treating the isomerized base oil has an average primary particle size of 250-2000 microns. In one embodiment, the FT base oil is first heated during pretreatment with the solid sorbent to a temperature of 50° C. to 300° C. In another embodiment, the pre-treatment is at a temperature of 50° C. to 120° C. In a third embodiment, an inert gas such as nitrogen is passed through the oil to help with the filtering process. In a fourth embodiment, the isomerized base oil is first pre-treated with a pass through a bauxite filtration process before being filtered through the clay or clay-like sorbent. In a fifth embodiment, the isomerized base oil is re-treated or re-run through the acid-activated clay bed if the properties do not meet USP/EP requirements after the first or second pass.

In one embodiment, the isomerized base oil is treated at the rate of 2,000-80,000 gallons of oil per ton of adsorbent, before the adsorbent is regenerated or replaced. In another embodiment, this rate is between 5,000-40,000 gallons per ton. In another embodiment, the isomerized base oil is treated at a space velocity less than 2 per hour. In yet another embodiment, the space velocity ranges from 0.05 to 1.5 per hour. In a fourth embodiment, the space velocity ranges from 0.10 to 1 per hour.

Additives used in formulating the compositions can be blended into the filtered base oil individually or in various sub-combinations. In one embodiment, all of the components are blended concurrently. In one embodiment, the composition is prepared by mixing the isomerized base oil matrix with the separate additives at an appropriate temperature, such as approximately 60° C., until homogeneous.

In another embodiment, the additives are added in stages, with the isomerized base oil and at least an extreme pressure additive being blended in a first stage; a non-toxic antioxidant/emulsifier compound added to the mixture to detoxify and emulsify the mixture so as to form a non-toxic second stage mixture. In another embodiment, the isomerized base oil, extreme pressure additive and antioxidant substituents in the second stage mixture is emulsified and neutralized to a pH range between 7.3 and 7.48 prior to the addition of an antimicrobial.

Properties: The medicinal white oil is odorless and tasteless. In one embodiment, the oil surpasses the requirements of FDA and USP for neutrality, sulfur compounds, solid paraffins, UV absorbance (CFR 178.3620(b)), and RCS. In one embodiment, the DBD/DBF (dioxin precursors) levels are below the 1 ppb Canadian limit for lubricant discharge into water and below the 0.5 ppb detection limit. In another embodiment, the medicinal white oil meets the requirements of at least one of European Pharmacopeia 3^{rd} edition and US Pharmacopeia 23^{rd} edition. In yet another embodiment, the medicinal white oil meets or surpasses the solid paraffin test specified in USP XX (1980) pp. 532-533.

In one embodiment, the medicinal white oil has a UV absorbance at 260 to 350 nm of less than 0.1. In a second embodiment the oil has a UV absorbance at 260 to 350 nm of less than 0.05. In a third embodiment the oil has a UV absorbance at 280 to 289 nm of less than 4. In a fourth embodiment the oil has a UV absorbance at 280 to 289 nm of less than 2.

In one embodiment, the medicinal white oil is inherently biodegradable. In a second embodiment, the medicinal white oil is readily biodegradable, with the OECD 301D (closed bottle test) level ranging from 30 to 93%. In one embodiment, a medicinal white oil with a kinematic viscosity at 40° C. of less than 10 mm²/s has an OECD 301D biodegradability of >90%. In a second embodiment, a medicinal white oil with a kinematic viscosity at 40° C. in the range of 10-15 mm²/s has an OECD 301D biodegradability of >=80. In a third embodiment, a medicinal white oil with a kinematic viscosity at 40° C. in the range of 30-40 mm²/s has an OECD 301D biodegradability of >=30.

In one embodiment, the medicinal white oil comprises a sufficient amount of thickener for the product to have a viscosity of >3 mm²/s at 100° C. and >15 mm²/s at 40° C.

In another embodiment, the medicinal white oil composition has a specific gravity in the range of 1.120 to 1.150, a flash point of 170-185° C., a pour point in the range of −5 to −20° C., a kinematic viscosity at 40° C. in the range of 10 to 75, a SUS viscosity at 100° F. in the range of 70 to 400, a Saybolt color of >30.

In yet another embodiment, the medicinal white oil composition has a SUS (Saybolt Universal Seconds) viscosity of >2000 at 40° C. and >400 at 100° C., a viscosity index of >300, and a pour point of <−10° C.

Applications: In one embodiment, the medicinal white oil composition can be used in the lubrication of all types of enclosed gear, chain guide, chain and conveyor applications where there is a chance of incidental contact with food, foodstuffs, drinking water, potable water or ground water may occur. These applications can typically be found in industries including plants wherein food, poultry, egg, fish, seafood, beverage, water treatment, plants, vegetables, fruit, dairy products, snack food, dry food, pet food, animal feed, or pharmaceutical products are concerned; equipment to be used in processing and/or manufacturing of health care products, dental instruments, dental care products, food and/or drink products, cosmetic and/or pharmaceutical products.

In one embodiment, the medicinal white lubricant oil composition is used for household applications including treatment of surfaces that may be in direct or indirect contact with food, beverages, and the like, e.g., cutting boards, adhesives, household cleaners, polishers, sprayers on oil pans, etc. In another embodiment, the medicinal oil is used to impregnate wrapping paper to keep food crisp, control foam in beet sugar and vinegar production, and enhance leather tanning process. In yet another embodiment, the medicinal oil is used to treat textile materials to impart soil release and stain resistant characteristics thereto. In another embodiment, as a plasticizer or as an extender for polymers, as an adhesive for food packaging, or as a caulk or sealant. In a fifth embodiment, in a paste formulation for seed treatment or a liquid formulation for foliage treatment (of plants).

In one embodiment, the medicinal white oil having a non-toxic antimicrobial incorporated within is used with dental tools and some medical devices, which are designed and/or required to come into contact with the human body and/or its internal parts or have a high probability of incidental contact with the body.

In yet another embodiment, the medicinal white oil is used directly with product intended to be digested, e.g., as a release agent, binder, and lubricant in or on capsules and tablets containing concentrates of flavoring, spices, condiments, and nutrients intended for addition to food, excluding confectionery; as a release agent, binder, and lubricant in or on capsules and tablets containing food for special dietary use; as a float on fermentation fluids in the manufacture of vinegar and wine to prevent or retard access of air, evaporation, and wild yeast contamination during fermentation; as a defoamer in food; in bakery products, as a release agent and lubricant; in dehydrated fruits and vegetables, as a release agent; in egg white solids, as a release agent; on raw fruits and vegetables, as a protective coating; etc.

In one embodiment of the medicinal white oil composition having a high viscosity is used in personal care/cosmetics/toiletry applications including skin care products requiring emolliency and skin protection such as baby oils, creams, lotions, massaging oil, suntan oils, sunscreens; lipstick, make-up, make-up remover; soaps; bath oils; hair conditioners, hair gels, and the like. In yet another embodiment, the medicinal white oil composition is used in pharmaceutical applications including laxatives and topical ointments.

The following Examples are given as non-limitative illustration of aspects of the present invention.

### EXAMPLES

Unless specified otherwise, the components in the examples are as follows:

Isomerized base oils used in examples 1-24 are FT base oils from Chevron Corporation of San Ramon, Calif. The PAO (poly alpha olefins)/synthetic base oils in the comparable examples 25-30 are either from Chevron Corporation or Conoco Phillips. The properties of the base oils used in examples 1-30 are shown in Tables 2 (PAO base oils) and 3 (FT base oils).

### Examples 1-30

Different FT base oils were passed over absorbent beds containing alumina or Tonsil 630G. The properties of Tonsil 630G are shown in Table 4. The properties of the oils were measured before and after passing through the beds. The properties were also compared with the properties of FT base oils without any filtration. The results are presented in Table 5. “TWO” means technical white oils according to specification FDA CFR 178-3620 (b). USP means USP white oils specifications described in United States Pharmacopoeia XX (1980). As shown, some of the FT base oils were successfully treated to meet USP requirements right after the first pass, with exceptional UV absorbance properties far surpassing the USP requirements. Some of the prior art base oils met TWO requirements after passing through the beds. However, none met USP requirements even after passing through the Tonsil 630G beds. Additionally as shown, acid-activated clay gave much better results than alumina as adsorbent.

### Examples 31-32

Two FT base oils samples (ABQ0049 and ABQ0060) as disclosed in US Patent Publication No. 2006/0016721 are passed over absorbent beds containing Tonsil 630G at a space velocity ranging from 0.1 to 1 per hour. The properties of the oils are measured after filtering. It is expected that the white FT base oils become medicinal grade after filtering to meet the requirements of FDA 21 CFR 172.878 and FDA 21 CFR 178.3620(a), United States Pharmacopeia (U.S.P.) XX (1980) at page 532 for readily carbonizable substances, and U.S.P. XVII at page 400 for sulfur compounds. The properties of the white base oil feeds used in Examples 31-32 are as listed in Table 1.

TABLE 1

| Properties | Feed Ex. 31 | Feed Ex. 32 |
|---|---|---|
| Kinematic Viscosity @ 40° C., mm2/s | 99.38 | 97 |
| Kinematic Viscosity @ 100° C., mm2/s | 14.84 | 14.57 |
| Viscosity Index | 156 | 156 |
| Cold Crank Viscosity @ −25° C., cP | 13,152 | 12,504 |
| Cold Crank Viscosity @ −20° C., cP | 6,767 | 6,462 |
| Pour Point, ° C. | −12 | −13 |
| Cloud Point, ° C. | 15 | 12 |
| n-d-m | | |
| Molecular Weight, gm/mol (VPO) | 697 | 702 |
| Density, gm/ml | 0.8317 | 0.8314 |
| Refractive Index | 1.4636 | 1.4638 |
| Paraffinic Carbon, % | 93.44 | 94.76 |
| Naphthenic Carbon, % | 6.56 | 5.24 |
| Aromatic Carbon, % | 0.00 | 0.00 |
| Oxidator BN, hrs | 35.27 | 38.01 |
| SULFUR | <1 | <1 |
| NITROGEN | <0.1 | <.1 |
| Noack, wt. % | 1 | 1 |
| Saybolt Color | 24 | 30.4 |
| Flash Point, ° C. | 210 | 284 |

TABLE 3

| | | | | W9884 | |
|---|---|---|---|---|---|
| | W9881 | W9882 | W8222 | Synthetic | W9935 |
| | PAO4 | PAO6 | PAO8 | 40 PAO | PAO100 |
| Kin Visc., cSt @ 40° C. | 17.02 | 30.66 | 46.55 | 405.3 | 1282 |
| Kin Visc., cSt @ 100° C. | 3.865 | 5.878 | 7.771 | 39.72 | 101.7 |
| VI | 121 | 139 | 136 | 147 | 168 |
| Pour Point, ° C. | — | — | (−57) | −36 | −33 |
| Mol. weight, gm/mol (VPO) | 428 | 505 | 587 | — | — |
| Density, gm/ml | 0.8148 | 0.8236 | 0.832 | — | — |
| Refractive Index | 1.4549 | 1.4594 | — | — | — |
| Oxidator BN, hrs | 40.18 | 35.05 | 24.15 | 29.34 | 32.44 |
| Noack, wt. % | 14.03 | 7.08 | 3.37 | 1.45 | 0.091 |
| HPLC-UV (LUBES) | | | | | |
| 1-Ring | 0.00368 | 0.0113 | 0 | 0.5537 | — |
| 2-Ring | 0.00036 | 0 | 0 | 0.0035 | — |
| 3-Ring | 0.00009 | 0 | 0 | 0 | — |
| 4-Ring | 0 | 0 | 0 | 0 | — |
| 6-Ring | 0 | 0 | 0 | 0 | — |
| Aromatics Total | 0.0413 | 0.0113 | 0 | 0.5572 | — |
| COC Flash Point, ° C. | 220 | 250 | 260 | 294 | 288 |

TABLE 4

| | |
|---|---|
| Bulk density, g/l | 500-600 |
| Free moisture (2 h, 110° C.), % max | <6% |
| Loss on ignition (2 h, 1000° C.), % max | <10% |
| pH (10% suspension, filtered) | 2.4-3.0 |
| Free Acidity, mg KOH/g | 2.7-3.3 |
| Total Acidity, mg KOH/g | 9.0-12.0 |
| Surface area, m²/g | 230-250 |
| Micropore volume (0-80 nm), ml/g | 0.30-0.35 |
| Particle size >90 wt % through 20 mesh | 850 μm |
| Particle size <10% through 60 mesh | 250 μm |

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items.

This written description uses examples to disclose the invention, including the best mode, and also to enable any person skilled in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

All citations referred herein are expressly incorporated herein by reference.

TABLE 2

| | GQ ID | | | | | |
|---|---|---|---|---|---|---|
| Sample | BST449 | BST450 | BST451 | W9917 | W9916 | W9918 |
| API | — | — | — | — | — | — |
| Aniline Point, ° F. | — | — | — | — | — | — |
| Kin. Visc. | 17.74 | 37.92 | 99.38 | 42.3 | 16.49 | 106.4 |
| @40° C., cSt | | | | | | |
| Kin. Visc. | 4.12 | 7.129 | 14.84 | 7.929 | 4.039 | 16.01 |
| @ 100° C., cSt | | | | | | |
| Viscosity Index | 138 | 153 | 156 | 162 | 150 | 161 |
| CCS Vis. | | | | 24,287 | 2,450 | — |
| @ −40° C., cP | | | | | | |
| CCS Vis. | 1,596 | 6,966 | | 10,149 | 1,335 | — |
| @ −35° C., cP | | | | | | |
| CCS Vis. | 941 | 3,771 | | 4,936 | | 46,991 |
| @ −30° C., cP | | | | | | |
| CCS Vis. | | 2,200 | 13,152 | 2,584 | | 18,905 |
| @ −25° C., cP | | | | | | |
| Pour Point, ° C. | −26 | −20 | −12 | −11 | −25 | −10 |
| Cloud Point, ° C. | −20 | −13 | 15 | −1 | −12 | 15 |
| n-d-m | | | | | | |
| Mol. Wt., gm/mol | 431 | 540 | 697 | 549 | 416 | 743 |
| (VPO) | | | | | | |
| Density, gm/ml | 0.8128 | 0.8222 | 0.8317 | 0.8241 | 0.8116 | 0.8330 |
| Refractive Index | 1.4541 | 1.459 | 1.4636 | 1.4596 | 1.4536 | 1.4641 |
| Paraffinic | 95.99 | 95.47 | 93.44 | 93.68 | | 92.98 |
| Carbon, % | | | | | | |
| Naphthenic | 4.01 | 4.53 | 6.56 | 6.32 | | 7.02 |
| Carbon, % | | | | | | |
| Aromatic Carbon, % | 0.00 | 0.00 | 0.00 | 0.00 | | 0.00 |
| Oxidator BN, hrs | 41.02 | 42.07 | 35.27 | 45.86 | 50.43 | 45.32 |
| SULFUR | <2 | <2 | <1 | — | — | — |
| Nitrogen | <0.1 | <0.1 | <0.1 | — | — | — |
| Noack, wt. % | 10.22 | 2.49 | 1 | 2.02 | 13.01 | 0.95 |
| Traction Coef. @ | — | — | — | — | 0.0241 | |
| 15 cSt | | | | | | |
| Film thick. @ | — | — | — | — | 198 | |
| 15 cSt, nm | | | | | | |
| Saybolt Color | — | — | 24 | — | 20.7 | |
| HPLC-UV Lubes | | | | | | |
| 1-Ring | 0 | 0 | — | 0.00414 | 0.0196 | 0.02737 |
| 2-Ring | 0 | 0 | — | 0.00124 | 0.0005 | 0.00325 |
| 3-Ring | 0 | 0 | — | 0 | 0 | 0 |
| 4-Ring | 0 | 0 | — | 0 | 0 | 0 |
| 6-Ring | 0 | 0 | — | 0 | 0 | 0 |
| Aromatics Total | 0 | 0 | — | 0.00538 | 0.0202 | 0.03062 |
| Flash Point, ° C. | 232 | 258 | 210 | — | — | — |
| SIMDIST TBP | | | | | | |
| (WT %), F. | | | | | | |
| TBP @0.5 | 732 | 805 | 879 | 832 | 418 | 915 |
| TBP @5 | 758 | 836 | 935 | 869 | 723 | 963 |
| TBP @10 | 770 | 850 | 963 | 884 | 741 | 988 |
| TBP @20 | 784 | 869 | 997 | 902 | 763 | 1011 |
| TBP @30 | 795 | 884 | 1021 | 916 | 780 | 1040 |
| TBP @40 | 805 | 897 | 1042 | 928 | 796 | 1057 |
| TBP @50 | 813 | 913 | 1060 | 940 | 812 | 1074 |
| TBP @60 | 822 | 930 | 1079 | 953 | 829 | 1092 |
| TBP @70 | 832 | 947 | 1099 | 971 | 847 | 1113 |
| TBP @80 | 843 | 973 | 1122 | 989 | 867 | 1141 |
| TBP @90 | 857 | 1004 | 1153 | 1006 | 887 | 1181 |
| TBP @95 | 867 | 1033 | 1175 | 1022 | 899 | 1213 |
| TBP @99.5 | 887 | 1078 | 1219 | 1056 | 921 | 1290 |
| FIMS | | | | | | |
| Saturates | 75.3 | 73.1 | 69.7 | 70 | 78.9 | 65.4 |
| 1-Unsaturation | 23.6 | 26.5 | 29.6 | 27.9 | 20.3 | 33.1 |
| 2-Unsaturation | 0.9 | 0.2 | 0.7 | 2 | 0.8 | 1.2 |
| 3-Unsaturation | 0.1 | 0 | 0 | 0 | 0 | 0.3 |
| 4-Unsaturation | 0 | 0 | 0 | 0 | 0 | 0 |
| 5-Unsaturation | 0 | 0 | 0 | 0 | 0 | 0 |
| 6-Unsaturation | 0.1 | 0.2 | 0 | 0.1 | 0 | 0 |
| Branching Index | 27.25 | 24.29 | 21.66 | 23.20 | 26.18 | 20.83 |
| Branching | 14.83 | 17.94 | 21.45 | 22.71 | 19.02 | 27.05 |
| Proximity | | | | | | |
| Alkyl Branches | 2.9 | 3.33 | 3.7 | 3.34 | 2.85 | 4.02 |
| per Mol. | | | | | | |
| Methyl Branches | 2.26 | 2.62 | 2.85 | 2.73 | 2.36 | 3.29 |
| per Mol. | | | | | | |
| FCI | 4.56 | 6.92 | 10.68 | 8.91 | 5.65 | 14.36 |
| Alkyl Branches/ | 9.42 | 8.63 | 7.43 | 8.53 | 9.58 | 7.58 |
| 100 Carbons | | | | | | |
| CH3 Branches | 7.35 | 6.79 | 5.73 | 6.97 | 7.94 | 6.20 |
| per 100 Carbons | | | | | | |
| % Olefins by | 0.32 | 1.38 | 2 | 0.00 | 0.00 | 0.00 |
| Proton NMR | | | | | | |
| FIMS 1-unsat- | | — | — | — | — | — |
| NMR Olefins | | | | | | |
| Multicycloparaffin | — | — | — | — | — | — |
| Mono/Multi ratio | — | — | — | — | — | — |

GQ ID

| Sample | P2488 | N9713 | W9836 | W9776 | W9780 | W9782 |
|---|---|---|---|---|---|---|
| API | 40.1 | 44.99 | 2055 | 2003 | 2083 | 2083 |
| Aniline Point, ° F. | 270.4 | | | | | |
| Kin. Visc. | 37.56 | 6.939 | 7.658 | 17.20 | 42.19 | 86.72 |
| @40° C., cSt | | | | | | |
| Kin. Visc. | 7.336 | 2.18 | 2.333 | 4.101 | 7.901 | 13.14 |
| @ 100° C., cSt | | | | | | |
| Viscosity Index | 165 | 123 | 124 | 145 | 161 | 152 |
| CCS Vis. | — | | — | 2,579 | 36,130 | — |
| @ −40° C., cP | | | | | | |
| CCS Vis. | — | <900 | — | 1,561 | 13,547 | — |
| @ −35° C., cP | | | | | | |
| CCS Vis. | — | — | — | 905 | 5,802 | — |
| @ −30° C., cP | | | | | | |
| CCS Vis. | — | — | — | — | 2,896 | 16,528 |
| @ −25° C., cP | | | | | | |
| Pour Point, ° C. | −20 | −37 | −46 | −20 | −14 | −4 |
| Cloud Point, ° C. | 17 | −28 | −22 | −11 | 2 | 18 |
| n-d-m | | | | | | |
| Mol. Wt., gm/mol | 506 | 324 | 314 | 428 | 575 | 724 |
| (VPO) | | | | | | |
| Density, gm/ml | — | 0.7973 | 0.8026 | 0.8137 | 0.8261 | 0.8326 |
| Refractive Index | — | 1.4461 | 1.4485 | 1.4541 | 1.4608 | 1.4642 |
| Paraffinic | — | 97.13 | 93.13 | 94.03 | 93.94 | 93.86 |
| Carbon, % | | | | | | |
| Naphthenic | — | 2.87 | 6.87 | 5.97 | 6.06 | 6.14 |
| Carbon, % | | | | | | |
| Aromatic Carbon, % | — | 0 | 0.00 | 0.00 | 0.00 | 0.00 |
| Oxidator BN, hrs | 24.08 | — | — | | 37.72 | 33.52 |
| SULFUR | — | — | — | — | — | — |
| Nitrogen | — | — | — | — | — | — |
| Noack, wt. % | — | 59.4 | — | 12.45 | 1.63 | 0.86 |
| Traction Coef. @ | — | — | — | — | 0.0169 | — |
| 15 cSt | | | | | | |
| Film thick. @ | — | — | — | — | 198 | — |
| 15 cSt, nm | | | | | | |
| Saybolt Color | — | — | +33.6 | — | — | — |
| HPLC-UV Lubes | | | | | | |
| 1-Ring | 0.0231 | — | — | 0.01553 | 0.00964 | 0.04479 |
| 2-Ring | 0.0045 | — | — | 0.00044 | 0.00106 | 0.00448 |
| 3-Ring | 0.0003 | — | — | 0 | 0 | 0 |
| 4-Ring | 0.0003 | — | — | 0 | 0 | 0 |
| 6-Ring | 0 | — | — | 0 | 0 | 0 |
| Aromatics Total | 0.0282 | — | — | 0.01596 | 0.0107 | 0.04927 |
| Flash Point, ° C. | — | — | 192 | — | — | — |
| SIMDIST TBP | | | | | | |
| (WT %), F. | | | | | | |
| TBP @0.5 | 695 | 599 | 583 | 332 | 838 | 906 |
| TBP @5 | 742 | 612 | 622 | 725 | 877 | 953 |
| TBP @10 | 777 | 620 | 636 | 756 | 890 | 974 |
| TBP @20 | 825 | 633 | 654 | 778 | 907 | 995 |
| TBP @30 | 858 | 647 | 667 | 795 | 920 | 1007 |
| TBP @40 | 883 | 660 | 678 | 810 | 930 | 1020 |
| TBP @50 | 906 | 672 | 688 | 824 | 939 | 1036 |
| TBP @60 | 928 | 685 | 697 | 836 | 948 | 1048 |
| TBP @70 | 950 | 697 | 706 | 849 | 959 | 1061 |
| TBP @80 | 973 | 709 | 715 | 861 | 973 | 1078 |
| TBP @90 | 995 | 722 | 727 | 876 | 987 | 1106 |
| TBP @95 | 1011 | 730 | 735 | 886 | 998 | 1140 |
| TBP @99.5 | 1031 | 745 | 753 | 905 | 1029 | 1228 |
| FIMS | | | | | | |
| Saturates | 72.8 | — | 72.7 | 72.9 | 55.3 | 42.7 |
| 1-Unsaturation | 27.2 | — | 19.3 | 22.2 | 34.6 | 39.4 |
| 2-Unsaturation | — | — | 3.9 | 3.1 | 8.1 | 10.3 |
| 3-Unsaturation | — | — | 2 | 1.0 | 1.9 | 5.2 |
| 4-Unsaturation | — | — | 1.7 | 0.4 | 0.0 | 1.9 |
| 5-Unsaturation | — | — | 0.5 | 0.0 | 0.0 | 0.4 |
| 6-Unsaturation | — | — | 0 | 0.4 | 0.0 | 0.0 |
| Branching Index | — | — | 30.21 | 26.88 | 23.09 | 20.12 |
| Branching | — | — | 14.05 | 18.08 | 23.40 | 28.02 |
| Proximity | | | | | | |
| Alkyl Branches | — | — | 2.17 | 2.89 | 3.36 | 3.89 |
| per Mol. | | | | | | |
| Methyl Branches | — | — | 1.90 | 2.39 | 2.77 | 3.26 |
| per Mol. | | | | | | |
| FCI | — | — | 3.15 | 5.53 | 9.61 | 14.49 |
| Alkyl Branches/ | — | — | 9.67 | 9.46 | 8.17 | 7.53 |
| 100 Carbons | | | | | | |
| CH3 Branches | — | — | 8.48 | 7.80 | 6.74 | 6.30 |
| per 100 Carbons | | | | | | |
| % Olefins by | — | — | 0.00 | 0.00 | 0.00 | 0.00 |
| Proton NMR | | | | | | |
| FIMS 1-unsat- | — | — | | 22.2 | 34.6 | 39.4 |
| NMR Olefins | | | | | | |
| Multicycloparaffin | — | — | | 4.9 | 10.0 | 17.8 |
| Mono/Multi ratio | — | — | | 4.5 | 3.5 | 2.2 |

TABLE 5

| | | | USP | USP | Saybolt | TWO | TWO | TWO | TWO | | | |
|---|---|---|---|---|---|---|---|---|---|---|---|---|
| | | | RCS | Solid | Color | UV (nm) | UV (nm) | UV (nm) | UV (nm) | USP UV (nm) | | |
| | Base oil | Absorbent | No. | Paraf. | TWO/USP | 280-289 | 290-299 | 300-329 | 330-350 | 260-350 nm | USP | TWO |
| Ex. | Type | type | <11.5 | Pass | +20/+30 | <4.0 | <3.3 | <2.3 | <0.8 | =<0.1 | spec | spec |
| 1 | BST449 | NONE | F/16.5 | P | +30 | 0.00000 | 0.00000 | 0.00106 | 0.00086 | 0.00106 @328 | Fail | Pass |
| 2 | BST449 | Tonsil630G | P/4 | P | +30 | 0.05153 | 0.05473 | 0.06377 | 0.06598 | 0.06598 @350 | Pass | Pass |
| 3 | W9917 | NONE | P/2.0 | P | +30 | 0.2385 | 0.18767 | 0.10647 | 0.01169 | 0.24403 @276 | Fail | Pass |
| 4 | W9917 | Alumina | P/2.0 | P | +30 | 0.47679 | 0.38469 | 0.23723 | 0.02052 | 0.48719 @276 | Fail | Pass |
| 5 | W9917 | Tonsil630G | P/1.0 | P | +30 | 0.23365 | 0.18378 | 0.10319 | 0.01529 | 0.23936 @276 | Fail | Pass |
| 6 | BST450 | NONE | P/5.0 | — | +30 | 0.01177 | 0.00999 | 0.00887 | 0.00715 | 0.01231 @276 | Pass | Pass |
| 7 | BST450 | Tonsil630G | P/4 | — | +30 | 0.01735 | 0.01269 | 0.01066 | 0.00825 | 0.02877 @260 | Pass | Pass |
| 8 | W9918 | NONE | P/3.0 | P | +20 | 0.03041 | 0.02762 | 0.01871 | 0.02197 | 0.06238 @260 | Fail | Pass |
| 9 | W9918 | Alumina | P/3.0 | P | +20 | 0.01160 | 0.01090 | 0.01604 | 0.01604 | 0.01604 @325 | Fail | Pass |
| 10 | W9918 | Tonsil630G | P/2.0 | P | +30 | 0.01125 | 0.00913 | 0.00660 | 0.00596 | 0.01125 @283 | Pass | Pass |
| 11 | W9782 | NONE | P/3.0 | F | −9 | 0.23228 | 0.18643 | 0.11942 | 0.01921 | 0.23835 @276 | Fail | Pass |
| 12 | W9782 | Alumina | P/2.0 | F | — | 0.03379 | 0.03054 | 0.02317 | 0.01065 | 0.03379 @281 | — | Pass |
| 13 | W9782 | Tonsil630G | P/1.0 | P | +30 | 0.00230 | 0.00243 | 0.00722 | 0.00718 | 0.00722 @324 | Pass | Pass |
| 14 | BST451 | NONE | F/18+ | P | +26 | 0.05132 | 0.04370 | 0.03923 | 0.03383 | 0.05348 @275 | Fail | Pass |
| 15 | BST451 | Tonsil630G | F/14 | P | +26 | 0.01739 | 0.01694 | 0.01678 | 0.01296 | 0.02937 @260 | Fail | Pass |
| 16 | N9713 | Tonsil630G | F/16 | P | +30 | — | — | — | — | 0.01986 @277 | Fail | — |
| 17 | P2488 | Tonsil630G | F/18+ | F | +23 | — | — | — | — | 0.37697 @280 | Fail | — |
| 18 | W9780 | Tonsil630G | P/2 | F | +30 | 0.0055 | 0.0003 | — | — | 0.01986 @282 | Fail | — |
| 19 | W9836 | Tonsil630G | F/16 | P | +30 | 0.0285 | 0.0218 | 0.0167 | 0.0067 | 0.37697 @275 | Fail | — |
| 20 | W9776 | Tonsil630G | P/2 | P | +30 | 0.0156 | 0.0098 | 0.0030 | 0.0002 | 0.01986 @282 | Fail | — |
| 21 | W9782 | Tonsil630G | P/2.5 | F | +30 | 0.0033 | 0.0003 | 0.0064 | 0.0000 | 0.37697 @283 | Fail | — |
| 22 | W9916 | Tonsil630G | P/3 | P | +30 | 0.04516 | 0.04169 | 0.03996 | 0.0393 | 0.01986 @282 | Pass | Pass |
| 23 | W9917 | Tonsil630G | P/3 | P | +30 | 0.01907 | 0.01712 | 0.01248 | 0.00875 | 0.37697 @283 | Pass | Pass |
| 24 | W9918 | Tonsil630G | P/3 | P | +25 | 0.09329 | 0.09053 | 0.07507 | 0.01630 | 0.01986 @285 | Fail | Pass |
| 25 | W9881 | Tonsil630G | F/17 | P | +30 | — | — | — | — | 0.01986 @328 | Fail | Pass |
| 26 | W9882 | Tonsil630G | F/17 | P | +30 | 0.0417 | 0.0340 | 0.0281 | 0.0257 | 0.37697 @276 | Fail | Pass |
| 27 | W8222 | Tonsil630G | F/18 | P | +30 | 0.2009 | 0.2040 | 0.1789 | 0.0191 | 0.01986 @292 | Fail | Pass |
| 28 | W9884 | Tonsil630G | P/10 | P | +30 | 2.7259 | 2.7196 | 2.3627 | 0.1630 | 0.37697 @289 | Fail | Fail |
| 29 | W9935 | Tonsil630G | P/6 | P | +28 | 3.3947 | 3.1626 | 2.9325 | 0.2666 | 2.39466 @281 | Fail | Fail |
| 30 | W9935 | Tonsil630G | P/5.5 | P | 28 | 3.4730 | 3.2758 | 3.1737 | 0.2974 | 3.47303 @284 | Fail | Fail |

1. Use of an isomerized base oil as a medicinal white oil, wherein the isomerized base oil is in compliance with at least one of European Pharmacopeia 3^{rd} Edition, US Pharmacopeia 23^{rd} edition (USP), FDA 21 CFR 172.878 and FDA 21 CFR 178.3620(a) for direct food contact, FDA 21 CFR 178.3620(b) for indirect food contact, and FDA 21 CFR 178.3570 (USDA H-1) specifications, wherein the isomerized base oil is filtered through a filter bed containing an acid activated clay having a surface area of at least 100 m²/g, and wherein the isomerized base oil has consecutive numbers of carbon atoms and less than 10 wt % naphthenic carbon by n-d-M.

2. The use according to claim 1, wherein the acid activated clay has a surface area of at least 150 m²/g.

3. The use according to claim 2, wherein the acid activated clay has a surface area of at least 200 m²/g.

4. The use of the an isomerized base oil in claim 1, in pharmaceutical/cosmetic/food applications.

5. The use of the an isomerized base oil in claim 1, wherein the filtered isomerized base oil is a technical white oil in compliance with FDA CFR 178-3620 (b).

6. The use of the an isomerized base oil in claim 5, wherein the filtered isomerized base oil is in compliance with US Pharmacopeia 23^{rd} edition (USP), meeting the USP RCS specification as measured according to ASTM 565-99, having a Saybolt color of greater than 30, passing the USP solid paraffin test, and having a UV absorbance at 260 to 350 nm of less than 0.1.

7. The use according to claim 1, wherein the isomerized base oil is a Fischer-Tropsch derived base oil made from a waxy feed.

8. The use according to claim 1, wherein the isomerized base oil has an average molecular weight between 600 and 1100, and an average degree of branching in the molecules between 6.5 and 10 alkyl branches per 100 carbon atoms.

9. The use according to claim 1, wherein the isomerized base oil has a ratio of weight percent molecules with monocycloparaffinic functionality to weight percent molecules with multicycloparaffinic functionality of greater than 15.

10. The use according to claim 1, wherein the isomerized base oil is made from a process in which the highly paraffinic wax is hydroisomerized using a shape selective intermediate pore size molecular sieve comprising a noble metal hydrogenation component, and under conditions of about 600° F. to 750° F. and wherein the isomerized base oil has a Noack volatility of less than 50 weight %.

11. The use according to claim 1, wherein the isomerized base oil has a kinematic viscosity at 100° C. of >1.808 mm²/s and a Noack volatility less than an amount calculated by: 1.286+20 (kinematic viscosity at 100° C.)⁻¹⁵+551.8 Ln (kinematic vicosity at 100° C.).

12. The use according to claim 1, wherein the isomerized base oil has a kinematic viscosity at 100° C. between 1.5 mm²/s and 36 mm²/s; a viscosity index greater than an amount calculated by the equation: Viscosity Index=28×Ln (the Kinematic Viscosity at 100° C.)+105; and a pour point of less than 0° C.

13. The use according to claim 1, wherein the isomerized base oil has between 5-18 wt. % molecules with cycloparaffinic functionality, less than 1.2 wt. % molecules with multicycloparaffinic functionality, a pour point less than 0° C., and a Saybolt color of at least 20.

14. A process for lubricating surfaces of equipment and parts for use in processing or packaging food, which method comprises the step of applying onto the surfaces of the equipment and parts a medicinal oil composition consisting essentially of an isomerized base oil that has been filtered through a filter bed containing an acid activated clay having a surface area of at least 100 m²/g, wherein the isomerized base oil has consecutive numbers of carbon atoms and has less than 10 wt % naphthenic carbon by n-d-M.

15. The process of claim 13, wherein the isomerized base oil is a Fischer-Tropsch derived base oil made from a waxy feed.

16. A process for preparing a medicinal oil, the process comprises the steps of:
providing an isomerized base oil having consecutive numbers of carbon atoms and has less than 10 wt % naphthenic carbon by n-d-M, a kinematic viscosity at 100° C. between 1.5 mm²/s and 36 mm²/s; a viscosity index greater than an amount calculated by the equation: Viscosity Index=28×Ln (the Kinematic Viscosity at 100° C.)+105; and a pour point of less than 0° C.;
filtering the isomerized base oil through a filter bed containing an acid activated clay having a surface area of at least 100 m²/g;
wherein the medicinal oil has a UV absorbance at 260 to 50 nm of less than 0.1.

17. The process of claim 16, wherein the isomerized base oil has a branching index less than or equal to 23.4, a branching proximity greater than or equal to 22.0, and a Free Carbon Index between 9 and 30.

18. The process of claim 16, wherein the isomerized base oil has at least 4 wt % naphthenic carbon.

19. The process of claim 16, wherein the isomerized base oil comprises greater than 10 wt. % and less than 70 wt. % total molecules with cycloparaffinic functionality, and wherein the isomerized base oil has a ratio of weight percent molecules with monocycloparaffinic functionality to weight percent molecules with multicycloparaffinic functionality greater than 15.

20. A lubricating oil meeting H-1 specifications for use in lubricating equipment and/or parts, the lubricating oil comprising;
a base fluid having a major portion of a U.S. Government approved oil for safe use in external or internal contact with the human body;
optionally up to 20 wt. % of at least an additive selected from the group of a food-grade thickener component, a food-grade antioxidant selected from the group of sterically hindered phenols and thiophenols, a food grade anti-rust additive, a non-toxic emulsifier, and a buffering agent;
wherein the base fluid is an isomerized base oil that has been filtered through a filter bed containing an acid activated clay having a surface area of at least 100 m²/g, and wherein the isomerized base oil has consecutive numbers of carbon atoms and has less than 10 wt % naphthenic carbon by n-d-M.

21. A medicinal white oil meeting at least one of European Pharmacopeia 3^{rd} Edition and US Pharmacopeia 23^{rd} edition (USP) for use in pharmaceutical/cosmetic/food applications, the medicinal white oil comprising:
an isomerized base oil that has been filtered through a filter bed containing an acid activated clay having a surface area of at least 100 m²/g for the isomerized base oil to have a UV absorbance at 260 to 50 nm of less than 0.1 and a Saybolt color of at least 30; and
optionally up to 20 wt. % of at least an additive selected from the group of a food-grade thickener component, a food-grade antioxidant selected from the group of sterically hindered phenols and thiophenols, a biological antioxidant; a non-toxic emulsifier, and a buffering agent.

22. The medicinal white oil of claim 21, wherein the medicinal white oil further comprises 0.001 to 2.0 wt. % of DL-alpha-Tocopherol (CAS 59-02-9) as an antioxidant.

23. The medicinal white oil composition of claim 21, wherein the composition is emulsified and neutralized to a pH range between 7.3 and 7.48.
